(19) United States
(12) Patent Application Publication
(10) Pub. No.: US 2006/0025346 A1
(43) Pub. Date: Feb. 2, 2006

(54) Permeability enhancer

(76) Inventors: Nobukazu Kashima, Kawasaki-shi (JP); Kiyonori Furuta, Kawasaki-shi (JP); Itsuya Tanabe, Kawasaki-shi (JP)

Correspondence Address: CERMAK & KENEALY LLP;ACS LLC, 515 EAST BRADDOCK ROAD, SUITE B, ALEXANDRIA, VA, 22314, US

(21) Appl. No.: 11/171,551

(22) Filed: Jul. 1, 2005

(30) Foreign Application Priority Data
Jul. 29, 2004 (JP) 2004-221815

(51) Int. Cl. A61K 38/04

(52) U.S. Cl. 514/19

(57) ABSTRACT

The present invention provides an enhancer for the absorption by membrane permeation of low molecular weight substances. An enhancer for the absorption of low molecular weight substances by membrane permeation comprising poly-γ-glutamic acid as an active ingredient, and a composition characterized by comprising poly-γ-glutamic acid and a functional food component or drug the permeability of which improves in the presence of poly-γ-glutamic acid, are provided.

### BACKGROUND OF THE INVENTION

1. Field of the Invention

The present invention relates to poly-γ-glutamic acid, which is useful for enhancing the absorption of low molecular weight substances.

### BRIEF DESCRIPTION OF THE RELATED ART

Various low molecular weight drugs are employed in the field of medicine. However, many of them are absorbed poorly by the intestines, precluding adequate utilization of their original medicinal effects and effectively narrowing the choices in the development of pharmaceutical products. Although some foods and beverages contain functional components that are useful for the human body (also referred to as “functional foods”), many of these components are also absorbed poorly by the intestines, precluding adequate utilization of their original functions. Although substances known as pharmaceutical product absorption enhancers exist, they tend to cause variation in the absorption of other agents with which they are employed in actual use. In practical terms, there are extremely few cases where such substances are added to pharmaceutical products. Accordingly, there is a need for substances which are able to safely enhance the intestinal absorption of such drugs, functional food components, and the like.

Sodium caprate was discovered to be an intestinal absorption enhancing substance during research of in vitro systems employing a Caco-2 cell monolayer. However, side effects due to excessive absorption of drugs and the like, and a risk of cell damage have been observed, due to a relatively strong ability to open tight junctions (Anderberg, E. K., et al., Pharm. Res., 10, 857-864 (1993); Tomita, M., et al., J. Pharmacol. Expr. Ther., 272, 739-743 (1995); Lindmark, T., et al., J. Pharmacol. Exp. Ther., 275, 958-964 (1995); Lindmark, T., et al., J. Pharmacol. Exp. Ther., 284, 362-369 (1998); Sakai, M., et al., J. Pharm. Pharmacol., 50(10), 1101-1108 (1998); and Utoguchi, N., et al., Pharm. Res., 15(6), 870-876 (1998)). Accordingly, there is a need for absorption enhancer that is both safe and relatively mild in its effect.

Poly-γ-glutamic acid is known as an absorption enhancer for minerals such as calcium. However, the absorption enhancement mechanism is related to the solubility of the mineral, and therefore the effect on the paracellular permeability is unknown (Japanese Patent Application Publication Nos. Heisei 3-30648 and Heisei 5-316999). Poly-γ-glutamic acid, an amino acid polymer in which D-glutamic acid and L-glutamic acid are mixed, is known to be the viscous component of natto, otherwise known as fermented soybeans, and is highly safe. Although Japanese Patent Application Publication No. 2002-257828 describes a general screening method for absorption enhancers employing Caco-2 derived from human colon cancer, there is no discussion of poly-γ-glutamic acid.

### SUMMARY OF THE INVENTION

It is an object of the present invention to provide a highly safe substance which is able to specifically enhance the permeability of low molecular weight functional components, that is, low molecular weight drugs and useful, low molecular weight functional food components, and the like, contained in foods and beverages.

The present inventors conducted extensive research into the above-stated problem and, as set forth below, discovered that poly-γ-glutamic acid is able to enhance the permeability of low molecular weight functional components and the like.

It is an object of the present invention to provide a pharmaceutical composition comprising a drug and poly-γ-glutamic acid, wherein said poly-γ-glutamic acid improves absorption of said drug when said composition is taken orally.

It is an object of the present invention to provide the pharmaceutical composition as described above, wherein said drug is a synthetic low molecular weight pharmaceutical product with a molecular weight not exceeding 1,000.

It is an object of the present invention to provide the pharmaceutical composition of as described above, wherein said drug is a weakly acidic anionic low molecular weight substance.

It is an object of the present invention to provide a food composition comprising a functional food component and poly-γ-glutamic acid, wherein said poly-γ-glutamic acid improves absorption of said functional food component when said composition is consumed orally.

It is an object of the present invention to provide the food composition as described above, wherein said functional food component has a molecular weight not exceeding 1,000.

It is an object of the present invention to provide the food composition as described above, wherein said functional food component comprises one or more components selected from the group consisting of monocarboxylic acids, flavonoids, vitamins, polyphenols, carotenoids, coenzymes, and physiologically active peptides.

It is an object of the present invention to provide a composition comprising a compound the permeability of which improves in the presence of 0 to 5 percent poly-γ-glutamic acid over permeability in the absence of poly-γ-glutamic acid when cells of the epithelial cell strain MDCK, derived from canine kidney collecting tubules, are cultured on a semi-permeable membrane to form a monolayer and the permeability of the membrane is measured; and poly-γ-glutamic acid.

It is an object of the invention to provide a method of enhancing the membrane permeability of a substance comprising administering a composition comprising said substance and poly-γ-glutamic acid.

It is a further object of the invention to provide the method as described above, wherein said substance is selected from the group consisting of a food and a drug.

It is a further object of the present invention to provide the method as described above, wherein said substance comprises a molecular weight not exceeding 1,000.

It is a further object of the present invention to provide the method as described above, wherein said drug comprises a weakly acidic anionic low molecular weight substance.

It is a further object of the present invention to provide the method as described above, wherein said food comprises one or more components selected from the group consisting of monocarboxylic acids, flavonoids, vitamins, polyphenols, carotenoids, coenzymes, and physiologically active peptides.

It is a further object of the present invention to provide the method as described above, wherein said substance comprises a compound the permeability of which improves in the presence of 0 to 5 percent poly-γ-glutamic acid over permeability in the absence of poly-γ-glutamic acid when cells of the epithelial cell strain MDCK, derived from canine kidney collecting tubules, are cultured on a permeable membrane to form a single cell layer and the permeability of the membrane is measured; and poly-γ-glutamic acid.

### BRIEF DESCRIPTION OF THE DRAWINGS

FIG. 1 shows a chart depicting the enhancing action of sodium caprate on the permeability of model substances. Sodium caprate was added in a proportion of 0.25 percent.

FIG. 2 shows a chart depicting the enhancing action of the poly-γ-glutamic acid of the present invention on the permeability of model substances. Poly-γ-glutamic acid was added in a proportion of 1 percent.

FIG. 3 shows a chart depicting the concentration dependence of poly-γ-glutamic acid on the permeability of the low molecular weight model substance Flu.

FIG. 4 shows a chart depicting the molecular weight dependence of poly-γ-glutamic acid on the permeability of the low molecular weight model substance Flu. Poly-γ-glutamic acid was added in a proportion of 1 percent.

FIG. 5 shows a chart depicting the enhancing action of the poly-γ-glutamic acid of the present invention on the permeability of the low molecular weight drug AV-010. Poly-γ-glutamic acid was added in proportions of from 0 to 2 percent for an AV-010 concentration of 0.9 mg/mL.

FIG. 6 shows a chart depicting the enhancing action of the poly-γ-glutamic acid of the present invention on the permeability of the low molecular weight drug risedronate. Poly-γ-glutamic acid was added in proportions of from 0 to 2 percent for a risedronate concentration of 0.9 mg/mL.

FIG. 7 shows a chart depicting the enhancing action of the poly-γ-glutamic acid of the present invention on the permeability of the functional food component ferulic acid. Poly-γ-glutamic acid was added in proportions of from 0 to 2 percent for a ferulic acid concentration of 0.9 mg/mL.

### DESCRIPTION OF THE PREFERRED EMBODIMENTS

The in vitro permeation system employing the cultured monolayer of the epithelial cell strain Caco-2 (referred to simply as “Caco-2 cells” hereinafter) derived from human colon cancer is commonly used as it is thought to reflect human intestinal absorption in vivo (Artursson P. et al., Biochem. Biophys. Res. Commun., 175(3), 880-885 (1991); Stewart B. H. et al., Pharm. Res., 12, 693-699 (1995)). Additionally, for several low molecular weight drugs, the permeability in in vitro systems employing Caco-2 cells has been reported to correlate with the permeability in in vitro systems employing the cultured monolayer of the epithelial cell strain MDCK (Madin Darby Canine Kidney cell line) derived from canine kidney collecting tubules (referred to simply as “MDCK cells” hereinafter), and excellent correlation is reported to exist between the permeability in these two systems and in vivo human intestinal absorption (Irvine J. D. et al., J. Pharm. Sci., 88(1), 28-33 (1999); Braun A. et al., Eur. J. Pharm. Sci., 11, Suppl. 2, S51-S60 (2000)). Accordingly, the permeability in in vitro systems employing MDCK cells is thought to accurately reflect in vivo absorption in the human intestine.

Accordingly, the present inventors investigated the effect of sodium caprate on the permeability in an in vitro system employing MDCK cells. As a result, it became clear that sodium caprate nonspecifically increased the permeability of both low molecular weight model substances (sodium fluorescein, molecular weight 376: Flu, Lucifer Yellow CH, molecular weight.457: LY) and high molecular weight model substances (FITC-labeled dextran, molecular weight 10,000: FD-10) that permeate via paracellular pathway. The present inventors pursued their investigation for various highly safe food-derived substances. An investigation of the permeability enhancing effect on the above Flu and FD-10 in in vitro systems employing the above MDCK cells resulted in the discovery that poly-γ-glutamic acid exhibited almost no increase in the permeability of the high molecular weight model substance FD-10, but exhibited a marked the increase of permeability of the low molecular weight model substance Flu. Using the poly-γ-glutamic acid as the permeability enhancer of the present invention permits the effective enhancement of the absorption of orally administered low molecular weight functional components, that is, low molecular weight drugs and useful functional food components contained in foods and beverages.

The poly-γ-glutamic acid employed in the present invention may be extracted from a viscous substance such as natto, or secreted extracellularly by a member of the genus Bacillus, such as bacillus natto. The levan in natto, or the secretions of bacillus natto, may also be employed without negative effect. Although the molecular weight of poly-γ-glutamic acid is not specifically limited, a molecular weight of from 5×10³ to 3×10⁵ is desirable to facilitate preparation and the like.

Methods exist to produce poly-γ-glutamic acid of a desired molecular weight, including the use of an acid or a special enzyme not present in the intestines to cleave the γ bond. Another method is culturing bacillus natto and causing it to secrete poly-γ-glutamic acid of desired molecular weight. Poly-γ-glutamic acid produced by either of these methods can be employed without negative effect.

Known methods of increasing the capacity to produce poly-γ-glutamic acid by microbial fermentation include culturing a low ammonia acid producing mutants of bacillus natto (Japanese Patent Application Publication No. Heisei 8-154616), culturing a microbe in a medium which contains soy sauce malt or an extract thereof, fermented soy sauce brew, or a mixture thereof (Japanese Patent Application Publication No. Heisei 8-242880), and culturing a variant lacking or having reduced enzyme activity for synthesis of glutamic acid (Japanese Patent Application Publication No. 2000-333690). Poly-γ-glutamic acid produced by any of these methods may be employed, and there is no specific limitation to poly-γ-glutamic acid produced by these methods in particular.

Poly-γ-glutamic acid is commonly obtained as a sodium salt. Although desirably in the form of a salt, including sodium salt, free poly-γ-glutamic acid may also be employed. The substance whose permeability is to be enhanced is not specifically limited; however, low molecular weight substances, particularly anionic low molecular weight substances having weakly acidic groups, are preferred. Compounds having physical properties similar to those of sodium fluorescein (chemical formula (1)), sodium ferulate (chemical formula (2)), (−)-9-[1′S, 2′R-bis[hydroxymethyl)cyclopropane-1′-yl]methylguanine (AV-010) (chemical formula (3)), sodium risedronate (chemical formula (4)), and the like—for example, compounds having similar distribution rate (log P, o/w) and molecular weight—are thought to undergo the absorption enhancing effect. The distribution rate is generally measured in a 1-octanol/water system, and can be readily determined by the known flask vibration method or the like.

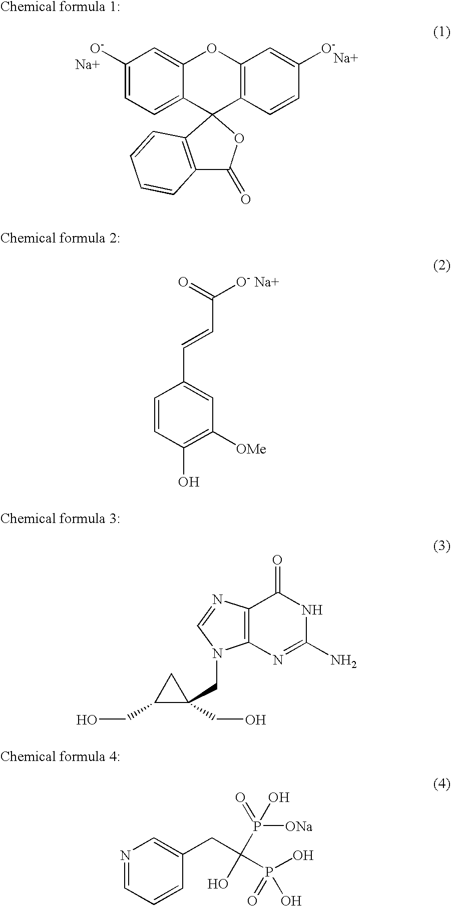

Functional food components the absorption of which is desirably enhanced include monocarboxylic acids, flavonoids, vitamins, polyphenols, carotenoids, and physiologically active peptides. Specific examples of these components include soybean isoflavone, cinnamic acid, vanillic acid, DHA, EPA (eicosapentaenoic acid), catechin, coenzyme Q, lutein, lignan (sesamins), soybean-decomposing peptides, and various herbal extracts; however, the present invention is not limited thereto. It is anticipated that of these compounds, those having molecular weights of about 1,000 or less, preferably 500 or less, will lend themselves to absorption increase. Furthermore, whether or not these functional food components and various pharmaceutical compounds currently on the market or under development are compounds the absorption of which can be enhanced by the present invention can be readily determined by methods similar to those set forth in examples 1 and 6 to 8 below. That is, based on the method of example 1, a determination is readily made by preparing MDCK cells and comparing the apparent permeability coefficient C (hereinafter referred to simply as “Papp”; cm/sec) of a test substance (the substance the permeability of which is being enhanced) in the presence of 0 to 5 percent poly-γ-glutamic acid to that in the absence thereof. Combining these functional food components and pharmaceutical product compounds with the highly safe poly-γ-glutamic acid of the present invention makes it possible to provide food and drug compositions with suitably enhanced absorption ratio.

The present invention will be further explained with reference to the following non-limiting examples.

### EXAMPLE 1

### Preparation of a Permeability Test Model System

MDCK cells were cultured for 3-4 days in a 37° C. carbonic acid gas incubator in a flask containing D-MEM/F12 culture medium (made by Gibco) containing 10 percent FBS. The cells were removed from the flask with Trypsin/EDTA solution (made by Gibco) and the individual cells were separated by pipetting. The cells were suspended in a culture medium identical to that described above, and 2×10⁵ cells/well were introduced in quantities of 0.1 mL to transwells (apical side) on a transwell plate (made by Costar). The transwells were then transferred to basal side wells containing 0.6 mL of the above medium and cultured for three days in a carbonic gas incubator at 37° C. After the cultures, the media in the apical side wells and in the basal side wells were replaced with fresh media identical to that set forth above. Incubation was then conducted under the same conditions as above for one day. This yielded a good MDCK cell monolayer.

When conducting an in vitro permeability test, the medium was removed from both wells, PBS solution (pH 6.5) was used to wash the apical side wells and PBS buffer solution (pH 7.4) was used to wash the basal side wells. Next, 0.1 mL of PBS buffer solution (pH 6.5) was added to the apical side wells and 0.6 mL of PBS buffer solution (pH 7.4) was added to the basal side wells. Incubation was then conducted in an incubator for 15 min at 37° C. to achieve equilibrium. The apical side wells were then emptied and refilled with a test substance prepared with PBS buffer solution (pH 6.5) and 0.1 mL of solution containing the test substance. Permeability test started at the point in time when these wells were moved to basal side wells that had been emptied and refilled with 0.6 mL of PBS buffer solution (pH7.4). Subsequently, sampling of the basal side wells was conducted over time. The quantity of the test substance in the sample collected in this manner was measured by fluorometric analysis and HPLC analysis, permitting determination of the amount of test substance undergoing permeation from the apical side to the basal side over the reaction period. This model system was employed in all of the following experiments.

### EXAMPLE 2

### The Effect of Sodium Caprate on the Permeability of Model Substances

A model substance prepared with PBS buffer solution (pH 6.5) (high molecular weight model substance FD-10, low molecular weight model substance Flu, or low molecular weight model substance LY) and sodium caprate prepared with the same buffer solution were added to the wells of the apical side. The final concentrations of FD-10, Flu, LY, and sodium caprate were 4.5 mg/mL, 0.09 mg/mL, 0.9 mg/mL, and 0.25 percent, respectively. The quantity of each model substance permeating from the apical side to the basal side at 37° C. was measured at reaction times of 30 to 120 minutes, and the Papp (cm/sec) was calculated according to the following equation.
Papp=dQ/dt×1/Co×1/A
wherein dQ/dt is the permeability rate (steady state flux, mol/sec), Co is the initial concentration in apical chamber (mol/ml) and A is the surface area of the porous membrane (cm2).

The degree of enhancement of absorption was denoted as a relative Papp for each reaction period, adopting one as the Papp when no sodium caprate was added. The results are given in FIG. 1, which shows that, even in an in vitro system employing MDCK cells, the sodium caprate exhibited a marked permeability enhancing effect on the high molecular weight model substance FD-10 and the low molecular weight model substances Flu and LY. The Papp of the individual model substances were calculated by placing 0.1 mL of sample collected from basal side wells in each well of a 96-well flat bottom plate (Costar, 3925) and measuring the fluorescence with a 485 nm excitation filter and 535 nm detection filter for Flu and FD-10, and a 405 nm excitation filter and 535 nm detection filter for LY, in a Wallac ARVO SX 1420 MULTILABEL COUNTER (identical below).

### EXAMPLE 3

### The Effect of Poly-γ-Glutamic Acid on Permeability of Model Substance

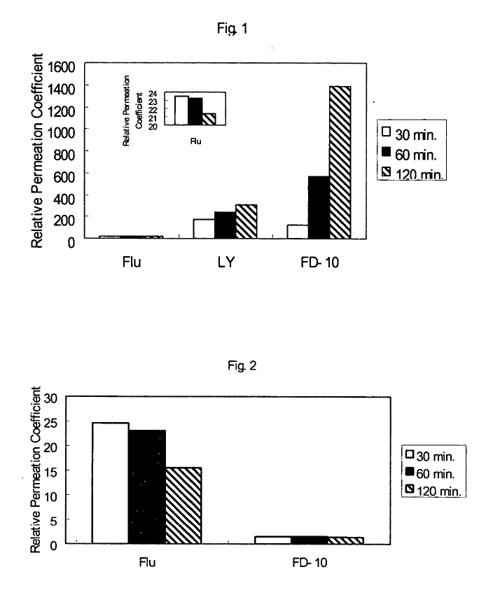

A model substance prepared with PBS buffer solution (pH 6.5) (high molecular weight model substance FD-10 or low molecular weight model substance Flu) and poly-γ-glutamic acid (molecular weight 26 K) prepared with the same buffer solution were added to the apical side. The final concentrations of FD-10, Flu, and poly-γ-glutamic acid were 4.5 mg/mL, 0.09 mg/mL, and 1 percent, respectively. The quantity of each model substance permeating from the apical side to the basal side at 37° C. was measured at reaction times of 30 to 120 minutes, and the Papp was calculated. The degree of permeability enhancement was denoted as a relative Papp for each reaction period, adopting 1 as the Papp when no poly-γ-glutamic acid was added. The results are given in FIG. 2, which shows that poly-γ-glutamic acid exhibited almost no enhancing effect on the permeability of the high molecular model substance FD-10, but exhibited a marked enhancing effect on the permeability of the low molecular weight model substance Flu. In contrast to the nonspecific and strong effect of sodium caprate indicated in example 2, the present invention exhibited a specific and mild effect on low molecular weight substances. Furthermore, this specificity might cause no, or extremely little, cell damage.

### EXAMPLE 4

### The Concentration Dependence of Poly-γ-Glutamic Acid on the Permeability of Low Molecular Weight Model Substance Flu

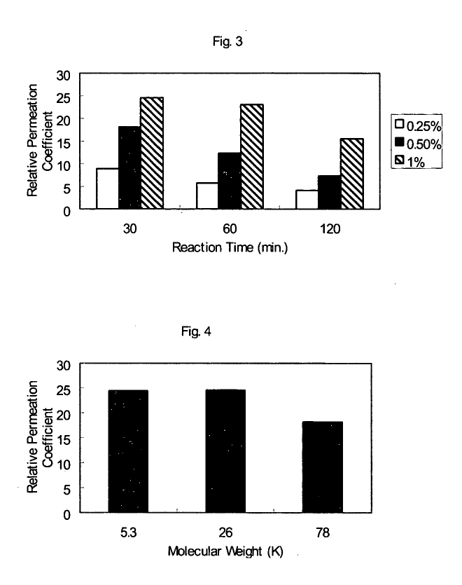

Low molecular weight model substance Flu and poly-γ-glutamic acid (molecular weight 26 K), both prepared with PBS buffer solution (pH 6.5), were added to the apical side to final concentrations of 0.09 mg/mL and 0.25 to 1 percent, respectively. The permeation amount of Flu from the apical side to the basal side at 37° C. was measured at reaction times of from 30 to 120 minutes and the Papp was calculated. The degree of permeability increase was denoted as a relative Papp for each reaction period, adopting 1 as the Papp when no poly-7-glutamic acid was added. The results are given in FIG. 3, which shows that the degree of the permeability increased in a manner dependent on the concentration added.

### EXAMPLE 5

### The Molecular Weight Dependence of Poly-γ-Glutamic Acid on the Permeability of Low Molecular Weight Model Substance Flu

Low molecular weight model substance Flu and poly-γ-glutamic acid with molecular weights of 5.3 to 78 K, both prepared with PBS buffer solution (pH 6.5), were added to the apical side to final concentrations of 0.09 mg/mL and 1 percent, respectively. The permeation amount of Flu from the apical side to the basal side at 37° C. was measured at a reaction time of 30 minutes and the Papp was calculated. The degree of permeability enhancement was denoted as a relative Papp for each reaction period, adopting 1 as the Papp when no poly-γ-glutamic acid was added. The results are given in FIG. 4, which shows that poly-γ-glutamic acid of all molecular weights markedly enhanced the permeability of Flu. Unless specifically stated otherwise, poly-γ-glutamic acid of a molecular weight of 26 K was employed in the embodiments set forth below.

### EXAMPLE 6

### The Effect of Poly-γ-Glutamic Acid on the Permeability of the Low Molecular Weight Drug AV-010

The low molecular weight drug AV-010, which is a drug having a specific antiviral action, was employed as the test substance.

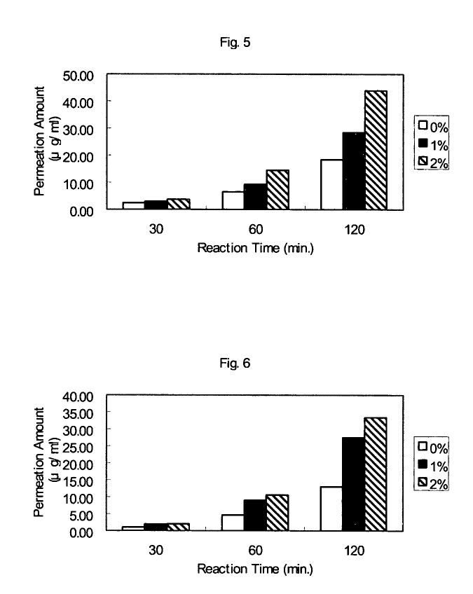

Low molecular weight drug AV-010 and poly-γ-glutamic acid, both prepared with PBS buffer solution (pH 6.5), were added to the apical side to final concentrations of 0.9 mg/mL and 1-2 percent, respectively. The permeation amount of AV-010 from the apical side to the basal side at 37° C. was measured at reaction times of 30 to 120 min. The amount of AV-010 permeation was determined by HPLC analysis on an Inertsil ODS-3 (G.L. Science) column using acetonitrile-containing acidic phosphate buffer solution as a mobile phase under conditions of a column temperature of 40° C. and a detection wavelength of 254 nm. The results are given in FIG. 5, which shows that poly-γ-glutamic acid markedly increased permeability of AV-010. This effect was dependent on concentration.

### EXAMPLE 7

### The Effect of Poly-γ-Glutamic Acid on the Permeability of the Low Molecular Weight Drug Sodium Risedronate (“Risedronate” Hereinafter)

The low molecular weight drug risedronate, not which is a compound having a specific antiosteoporotic effect, was employed as the test substance. Low molecular weight drug risedronate and poly-γ-glutamic acid, both prepared with PBS buffer solution (pH 6.5), were added to the apical side to final concentrations of 0.9 mg/mL and 1 to 2 percent, respectively. The amount of risedronate permeation from the apical side to the basal side at 37° C. was measured at reaction times of 30 to 120 min. The amount of risedronate permeation was determined by HPLC analysis on an Inertsil ODS-3 (G.L. Science) column using acetonitrile-containing acidic phosphate buffer solution as a mobile phase under conditions of a column temperature of room temperature and a detection wavelength of 263 nm. The results are given in FIG. 6, which shows that poly-γ-glutamic acid markedly enhanced the permeability of risedronate. This effect was concentration-dependent.

### EXAMPLE 8

### The Effect of Poly-γ-Glutamic Acid on the Permeability of Ferulic Acid

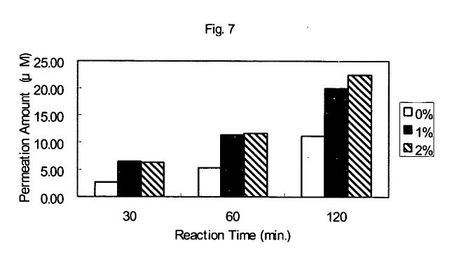

Sodium ferulate (“ferulic acid” hereinafter), which is a functional component useful to the human body that is found in specific foods and beverages, was employed as the test substance. Ferulic acid and poly-γ-glutamic acid, both prepared with PBS buffer solution (pH 6.5), were added to the apical side to final concentrations of 0.9 mM and 1 to 2 percent, respectively. The amount of ferulic acid permeation from the apical side to the basal side at 37° C. was measured at reaction times of 30 to 120 min. The amount of ferulic acid permeation was determined by HPLC analysis on an Inertsil ODS-3 (G.L. Science) column using acetonitrile-containing acidic phosphate buffer solution as a mobile phase under conditions of a column temperature of room temperature and a detection wavelength of 283 nm. The results are given in FIG. 7, which shows that poly-γ-glutamic acid markedly increased the permeability of ferulic acid. This effect was dependent on concentration.

The above examples show that poly-γ-glutamic acid enhances by 1 to 2.5-fold the permeability of not just low molecular weight model substances, but various low molecular weight drug and functional food components. As set forth above, a permeability in in vitro systems employing MDCK cells is thought to be a good reflection of in vivo absorption in the human intestine. Thus, a greater feasibility for use of these substances in the human body is indicated by poly-γ-glutamic acid.

While the invention has been described with reference to preferred embodiments thereof, it will be apparent to one skilled in the art that various changes can be made, and equivalents employed, without departing from the scope of the invention. Each of the aforementioned documents, including the foreign priority document JP 2004/221815, is incorporated by reference herein in its entirety.

We claim:

1. A pharmaceutical composition comprising a drug and poly-γ-glutamic acid, wherein said poly-γ-glutamic acid improves absorption of said drug when said composition is taken orally.

2. The pharmaceutical composition of claim 1, wherein said drug comprises a synthetic low molecular weight pharmaceutical product with a molecular weight not exceeding 1,000.

3. The pharmaceutical composition of claim 1, wherein said drug comprises a weakly acidic anionic low molecular weight substance.

4. A food composition comprising a functional food component and poly-γ-glutamic acid, wherein said poly-γ-glutamic acid improves absorption of said functional food component when said composition is consumed orally.

5. The food composition of claim 4, wherein said functional food component comprises a molecular weight not exceeding 1,000.

6. The food composition of claim 4, wherein said functional food component comprises one or more components selected from the group consisting of a monocarboxylic acid, a flavonoid, a vitamin, a polyphenol, a carotenoid, a coenzyme, and a physiologically active peptide.

7. A composition comprising:
a compound the permeability of which improves in the presence of 0 to 5 percent poly-γ-glutamic acid as compared to said compound's permeability in the absence of poly-γ-glutamic acid when cells of the epithelial cell strain MDCK are cultured on a permeable membrane; and
poly-γ-glutamic acid.

8. A method of enhancing the membrane permeability of a substance comprising administering a composition comprising said substance and poly-γ-glutamic acid.

9. The method of claim 8, wherein said substance is selected from the group consisting of a food and a drug.

10. The method of claim 8, wherein said substance comprises a molecular weight not exceeding 1,000.

11. The method of claim 9, wherein said drug comprises a weakly acidic anionic low molecular weight substance.

12. The method of claim 9, wherein said food comprises one or more components selected from the group consisting of a monocarboxylic acid, a flavonoid, a vitamin, a polyphenol, a carotenoid, a coenzyme, and a physiologically active peptide.

13. The method of claim 8, wherein said substance comprises a compound the permeability of which improves in the presence of 0 to 5 percent poly-γ-glutamic acid as compared to said compound's permeability in the absence of poly-γ-glutamic acid when cells of the epithelial cell strain MDCK are cultured on a permeable membrane.
